(19) United States
(12) Patent Application Publication
(10) Pub. No.: US 2020/0281686 A1
(43) Pub. Date: Sep. 10, 2020

(54) SURGICAL SITE BARRIER

(71) Applicant: David Finkelstein, St. Louis, MO (US)

(72) Inventor: David Finkelstein, St. Louis, MO (US)

(21) Appl. No.: 16/811,498

(22) Filed: Mar. 6, 2020

Related U.S. Application Data
(60) Provisional application No. 62/815,282, filed on Mar. 7, 2019.

(51) Int. Cl. A61B 90/40; A61B 90/30; A61B 90/50; A61B 46/20; A61B 46/00

(52) U.S. Cl. CPC A61B 90/40; A61B 90/30; A61B 2090/401; A61B 46/20; A61B 46/40; A61B 90/50

(57) ABSTRACT

A surgical site barrier which is smaller and provides for a sanitized area surrounding a surgical site or other wound, but which does not necessarily encompass the entire room or patient. Device is designed to site in close proximity to the patient and provide a frame with an opening where objects passing through the opening will first pass through an electromagnetic radiation (EMR) light wall and/or through an outward directed laminar airflow before being able to contact the wound.

### CROSS-REFERENCE TO RELATED APPLICATIONS

This application claims the benefit of U.S. Provisional Patent Application Ser. No.: 62/815282, filed Mar. 7, 2019, the entire disclosure of which is herein incorporated by reference.

### BACKGROUND OF THE INVENTION

### Field of the Invention

This disclosure is related to the field of barriers for use in the inhibition of infectious agents entering a surgical site or open wound.

### Description of the Related Art

Surgery is an inherently risky activity. Outside of the dangers associated with complications from the specific operation being performed, surgery typically requires at least small incisions through the skin. It is well known that any break in the skin is subject to potential infection as the skin is designed to resist invasion by microorganisms that can cause infection. Such risk is present in any open wound, but because surgery inherently and purposefully causes such a wound, the concern is in many respects greater as the risk can be avoided or reduced with proper protections in place. Wounds caused outside of surgery also often require surgical or other medical treatment to heal. For example, amputations, large cuts, compound fractures and other medical conditions commonly involve large wounds in the skin which require medical intervention to reduce the chance of death or additional injury. These treatments also present a risk of infection if not handled in the correct manner.

Danger from surgery related to the surgical site being the source of infection can present a major problem. Surgical Site infections (SSIs) and the broader Healthcare Associated Infections (HAIs) result in large numbers of deaths and serious follow on injuries around the world every year. In developed countries, such as the United States, the rates of surgical site infection are relatively low with the World Health Organization (WHO) estimating the numbers to be only around 1%. However, particularly in developing countries, the rates can be much higher. It has been found that rates of SSIs in the teens are not uncommon in many countries including some developed countries for certain types of surgeries and many SSIs, again particularly in developing countries, can be hard to treat and can lead to debilitating follow on health effects and even death.

The mechanisms to inhibit SSI development are straightforward and relatively simple. They simply involve disinfection and cleaning of everything that can contact the wound so that no infection vectors are present at the incision or wound site. Common methods are typically well-known to anyone who has ever watched a medical drama on television. Surgeons and other staff are dressed in specific attire which has been thoroughly cleaned and sanitized prior to use. Further, operating rooms are maintained in a highly clean state and are comprised of surfaces which are smooth and designed to aid in cleaning chemicals contacting microorganisms for disinfection. Further, surgical instruments which can enter the body are cleaned and sterilized prior to use.

Even the patient's skin is cleaned and disinfected prior to the incision so as inhibit any microorganisms residing on the skin from entering the incision area. Hospital cleaning procedures also commonly utilize strong antimicrobial agents (as opposed to antibacterial agents) to insure that all microorganisms are destroyed regardless of form and to reduce the formation of resistant pathogens.

Other more advanced forms of protection have also been proposed. For example, U.S. Pat. No. 9,949,881, the entire disclosure of which is herein incorporated by reference, provides a system which utilizes a non-turbulent (laminar) lateral airflow across the patient to inhibit pathogen entry.

While these methods have proven very effective at reducing SSI incidence as evidenced by the US rates of SSIs, they do so at a literal cost. Cleaning and sterilization methods are often very expensive and need to be very thorough. Further, they commonly take a not insubstantial amount of time to perform. Thus, the amount of patients that can be handled in any operating room is often limited by the time it takes to clean the room and instruments as well as cleaning and preparing the staff that will be in the surgery. This is particularly true with regards to more major operations where the risk of infection being transmitted is even higher, residence times in an operating room are higher, and the incisions and wounds are often larger.

While typical American operating rooms and procedures provide excellent disinfection, it is simply impossible to institute appropriate sanitation techniques outside of these carefully controlled environments. Many medical care facilities in the developing world lack the ability to provide operating rooms where air entering the room can be scrubbed and cleaned and personnel and tools entering the room can be controlled and disinfected. Many surgeries even have to be performed outdoors in some areas due to temperature concerns. Further, military operations and mass disaster situations can also present problems even in places where traditional clean operating rooms are available. In these situations, the number of patients who may require quick action such as surgery or even existing wound care to prevent death or major injury can rapidly swamp medical facilities' ability to maintain the operating rooms, personnel, and tools, in the necessary disinfected state.

In the event of large disasters, there can be too many patients in need of immediate surgery or other wound care than the cleaning and decontaminating procedures of a hospital can handle. In these situations, patients may need to be operated on using virtually any available surface. Similarly, patients may require immediate procedures on-site at a disaster area in order to stabilize them for transport to a hospital. This can result in procedures and surgeries having to be perfoulied outdoors or under other non-sanitized situations. Further, in military operations, wounded soldiers may require immediate medical attention where they are which can be under incredible unsanitary conditions.

### SUMMARY OF THE INVENTION

The following summary of the invention is provided to give the reader a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented in a later section.

Because of these and other problems in the art, there is a need for surgical site barrier which is smaller, may be portable, and provides for a sanitized area surrounding a surgical site or other wound, but which does not necessarily encompass the entire room or patient.

In an embodiment the device is designed to sit in close proximity to the patient and provide a frame with an opening where objects passing through the opening will first pass through an electromagnetic radiation (EMR) light bath and/or through an outward directed laminar airflow before being able to contact the wound.

Described herein, among other things, is a surgical site barrier comprising: a frame, the frame having an outer peripheral edge and an inner peripheral edge, the inner peripheral edge defining an opening through the frame; a source of electromagnetic radiation (EMR) positioned on the frame; and a drape positioned along the outer peripheral edge of the frame; wherein, the source of EMR produces a generally planar illumination forming a light wall across the opening.

In an embodiment of the barrier, the light wall is within the opening.

In an embodiment of the barrier, the light wall is below the opening.

There is also described herein, in an embodiment, a surgical site barrier device comprising: a frame, the frame having an outer peripheral edge and an inner peripheral edge, the inner peripheral edge defining an opening through the frame; a plurality of air projectors positioned on the frame; and a drape positioned along the outer peripheral edge of the frame; wherein, the air projectors produce intersecting generally laminar airflows which are non-parallel to the major dimension of the frame.

In an embodiment of the barrier, the airflows intersect over the opening.

In an embodiment of the barrier, the airflows intersect outside the inner periphery of the frame.

In an embodiment, the barrier further comprises an airflow from the frame which passes under an edge of the drape.

There is also described herein, in an embodiment, a surgical site barrier system comprising: a patient having an incision site; a surgical site barrier device positioned over the incision site, the surgical site barrier comprising: a frame, the frame having an outer peripheral edge and an inner peripheral edge, the inner peripheral edge defining an opening through the frame; a plurality of air projectors positioned on the frame; a source of electromagnetic radiation (EMR) positioned on the frame; and a drape positioned along the outer peripheral edge of the frame; wherein, the source of EMR produces a generally planar illumination forming a light wall across the opening; and wherein, the air projectors produce intersecting generally laminar airflows which are non-parallel to the major dimension of the frame.

In an embodiment of the system, the light wall is within the opening.

In an embodiment of the system, the light wall is below the opening.

In an embodiment of the system, the airflows intersect over the opening.

In an embodiment of the system, the airflows intersect outside the inner periphery of the frame.

In an embodiment, the system further comprises an airflow from the frame and under an edge of the drape.

In an embodiment, the system further comprises an adjustable arm attached to the frame at a proximal end, the adjustable arm positioning the frame over the incision site.

In an embodiment of the system, air and electricity for the air projectors and EMR is provided via the arm.

In an embodiment, the system further comprises an air compressor located at a distal end of the ann, opposite the proximal end, for suppling air to the air projectors.

In an embodiment of the system, the supplied air is filtered prior to being supplied to the air projectors.

In an embodiment of the system, the arm is connected to an alternating current (AC) power source.

In an embodiment, the system further comprises a direct current (DC) power source.

In an embodiment of the barriers and/or system, the EMR comprises UVC light, UV vacuum light, and/or UV light with a wavelength of less than 210 nanometers.

### BRIEF DESCRIPTION OF THE DRAWING

FIG. 1 shows a top view of an embodiment of a surgical site barrier.

FIG. 2 shows a side view of an embodiment of a surgical site barrier with airflow directed outwardly.

FIG. 3 shows the same view as FIG. 2 but with the airflow directed inwardly.

FIG. 4 shows a top view of an embodiment of a surgical site barrier in place over the torso of a patient.

FIG. 5 shows a side view of FIG. 4.

FIG. 6 provides a top view of another embodiment of a surgical site barrier.

FIG. 7 provides a side view of the embodiment of FIG. 6.

FIG. 8 provides a side view of an embodiment of a surgical site barrier which is designed to be portable and can fold to be used as a portable disinfection apparatus.

FIG. 9 provides a top view of the embodiment of FIG. 8.

FIG. 10 provides the folded view of FIG. 8 which the lights aimed downward into the page.

### DETAILED DESCRIPTION OF PREFERRED EMBODIMENTS

The present systems and methods relate to electomechanical barriers which inhibit the entry of microorganisms into a wound and particularly into a purposeful incision in the nature of a surgical wound.

Throughout this disclosure the ten is “wound” and “incision” may be used interchangeably as an incision is effectively a purposeful wound typically to provide for other kinds of care and specifically surgical intervention. The present systems are designed to isolate any type of current or future wound area from the surrounding environment and present disinfecting barriers to objects which would enter this site. Further, it should be recognized that terms such as “disinfected”, “sanitized”, “cleaned”, and “sterilized” typically have slightly different meanings from each other and often specify how “clean” something is based on the survival rates of certain organisms of interest. In the present disclosure, those terms are used interchangeably and are not intended to mean any particular level of pathogen or microorganism reduction.

This is because it should be recognized that no form of sanitization or sterilization is perfect and no form of site barrier can completely eliminate the possibility of infection. Thus, when the present disclosure refers to a surface or area as “disinfected” or “sanitized”, for example, it does not mean that there are no organisms which could cause infection present. Instead, the systems and methods proposed herein are designed to reduce the incidence of infection of a wound in the area or in contact with the object compared to if the barrier was not present. This will particularly be the case where surgery, or wound care, needs to be performed under less than ideal circumstances so that tools used in the surgery and the air surrounding the wound are less clean than could be obtained under better circumstances.

It should be recognized that the surgical site barriers contemplated herein are typically designed to be used when other, more advanced sanitization systems are not available. This will commonly be when surgery or other wound care needs to be performed outside of a typical American, or other major developed country, operating room. However, the surgical site barriers contemplated herein may be used in conjunction with more traditional operating room materials as desired for additional disinfection.

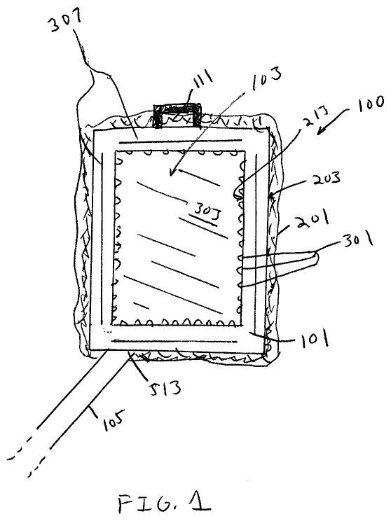

FIG. 1 provides for a first embodiment of a surgical site barrier (100) as viewed from the top. The barrier (100) comprises an exterior frame (101) which will typically be sized and shaped so as to sufficient to surround the size of any necessary incision with which the barrier (100) will be used. It should be recognized that barriers (100) of different sizes may therefore be provided for different procedures as it is preferred that while the frame (101) is larger in its dimensions than the incision opening will be, it is also preferred that the frame (101) only be as large as is necessary to access the incision (403) (as shown in FIG. 4) in the manner necessary for the surgery to be performed.

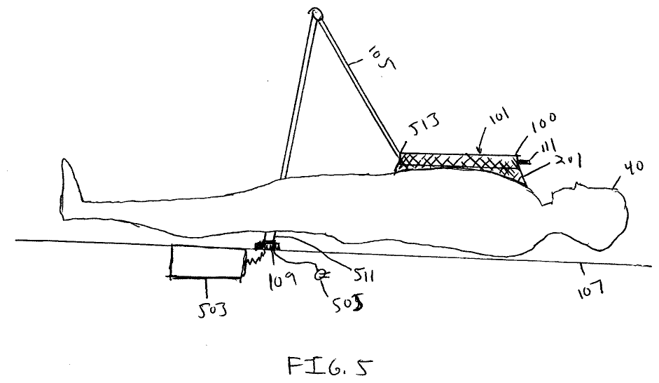

Generally, the frame (101) will also be large enough to readily admit the surgeon's hands and any tools necessary for the procedure through its enclosed opening (103). For certain surgeries (e.g. laparoscopic or robotic procedures) the frame (101) may be smaller as there is no need for a surgeon's hands to enter the opening (103). The frame (101) will typically be positioned above the patient (401) and directly above the incision (403) site as shown in FIGS. 4 and 5. It is preferred that the frame (101) be positioned as close to the patient (401) as possible as that there is no need for a surgeon to have to contort their arms or hands to reach the incision and the patient's internal structure through the frame (101).

To provide for the positioning, the frame (101) in an embodiment may be mounted on an adjustable arm (105) which may be mounted to a table or other surface (107) upon which the patient is positioned. This arm (105) may be permanently mounted to the surface (107) such as when the table is regularly used for operations, or may be designed to be temporarily attached through the a connector (109) such as, but not limited to, a clamp, suction cup, bolt, or other mechanisms as understood by one of ordinary skill in the art. The arm (105) may be designed to fold up to make the device (100) portable. The ann (105) and frame (101) will typically be constructed of a strong durable material which is easily cleaned such as, but not limited to, hard plastic or metal.

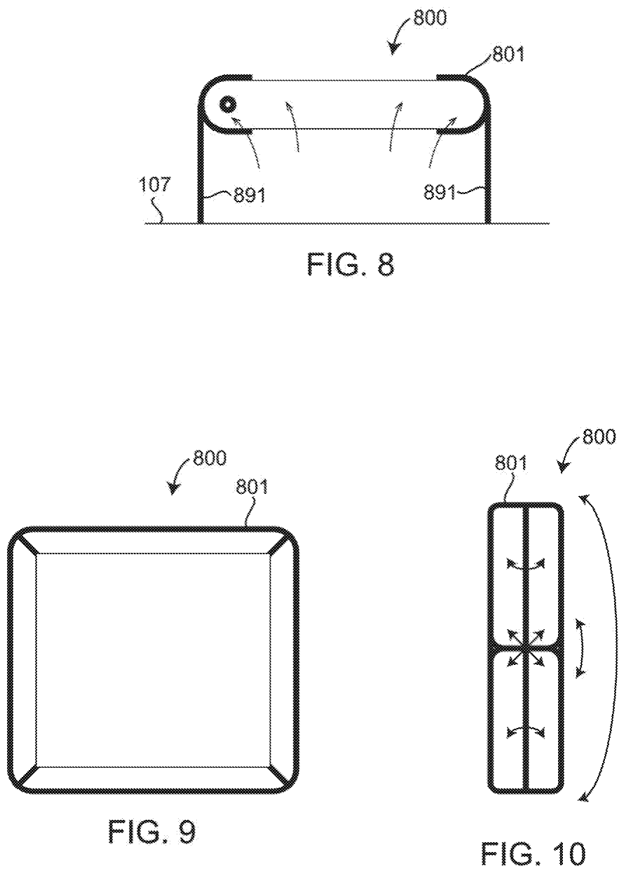

In an embodiment the arm (105) is eliminated and the frame (101) may be positioned directly on or over the patient through the use of rotatable legs (891) or similar devices. An embodiment of such a device (800) such as this is shown in FIGS. 8-10 These may be designed to rest on a surface (107) underlying the patient (401) (such as if the device need to be used on the floor for example) or may rest directly on the patient (401). If the device (100) is to be used directly on the patient (401), the frame (101) may actually be designed to be attached directly to the patient (401) and use the patient (401) for support. For example, this may mean the frame (101) includes straps, tape, or even direct adhesive to allow it to be held in place on the patient (401).

Further, while the frame (101) depicted in the various FIGS. is generally rectilinear in shape, this is by no means required and other shapes including, but not limited to, circles, ovals, and hexagons may be used in other embodiments. The frame (101), if it is designed to be moveable relative to the patient (401) during the operation will typically include at least one handle (111) mounted thereon to allow for the frame (101) and arm (105) to be moved during the operation. Handles (111) may alternatively or additionally be provided to aid in transport of the device (100).

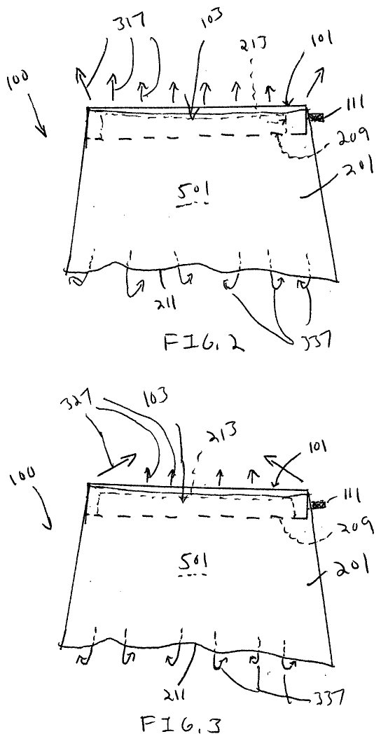

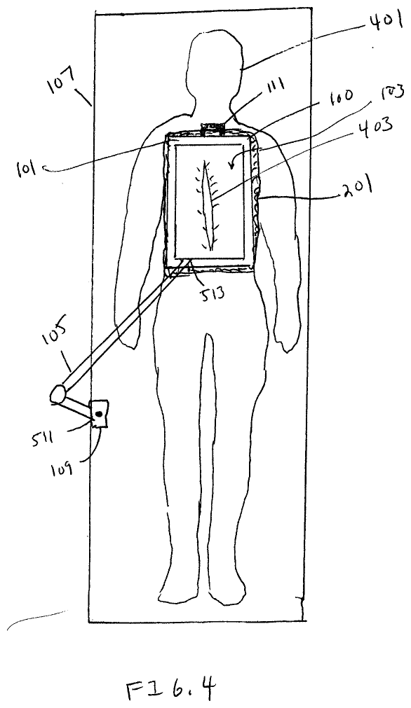

In an embodiment, below the frame (101), as is best visible in FIGS. 2 and 3, there will typically be a drape (201) or other similar structure that is designed to conform to the patient's topology under or nearly under the frame (101). The drape (201) will typically be designed of a smooth material designed to be sanitized and or sterilized prior to use. It will also often be weighted or of sufficient weight for it to pull itself relatively smooth under the force of gravity. The drape (201) will typically be mounted to the outside perimeter (203) of the frame (101) so that when the frame (101) is placed over the patient (401) the drape (201) will hang downward on the outside of the frame (101) between the frame (101) and the patient (401) as is best seen in FIG. 5.

The system (100) will typically provide for two forms of disinfection of the wound area. The first of these is that the frame (101) will typically include a plurality of electromagnetic radiation (EMR) projectors which are referred to herein as EMR radiators (301). EMR, as used herein, is intended to mean any form of EMR which may be useful for disinfection. This can include various wavelengths of light as well as wavelengths outside that realm such as radio waves. The wavelength of these EMR radiators (301) will typically be selected to be sufficient to kill most known microorganisms which present a danger to humans via wound transmission and will often be in the ultraviolet (UV) range. They may be of such wavelength that the EMR radiators (301) will damage human skin on contact (such as UVA and/or UVB light), or may not (such as is believed to be the case with certain forms of UVC light) depending on embodiment. If the lights (301) are capable of damaging human skin, doctors utilizing the barrier (100) will typically be wearing gloves and will therefore select gloves which can protect them from the particular EMR exposure. The lights (301) will often be in the form of a large number of light emitting diodes (LEDs) (301) which may be arranged on the underside (209) of the frame (101) or the inner periphery (213) of the frame (101) as shown in FIG. 1.

While the EMR radiators (301) will typically utilize ultraviolet light, it should be recognize that other forms of disinfecting waves and/or particles could also be transmitted by the EMR radiators (301) or in addition to the EMR radiators (301). This includes more traditional “radiation” such as X-rays and gamma rays as well as particles such as alpha and beta particles.

The EMR radiators (301) will typically be positioned in or on the frame (101) so as to strongly illuminate across the opening (103) in the frame (101) which a generally constant and unbroken plane (303) of EMR. This generally planar surface is referred to herein as a “light wall” (303), even though it may not include “light” in the traditional definition. The light wall (303) will generally be either within the inner periphery (111) of the frame (101) or just below it. In any case, it will typically be arranged above the patient (401) so that the strongest EMR is not directly incident on the incision (403) which the frame (101) is positioned over.

It is preferred that the EMR radiators (301) be strongly directional and may be shielded to so as to provide illumination across the opening (103) forming a generally planar light wall (303), but with relatively little light being projected directly above the frame (101) or downward into the patient (401). Such scattering may, however, be unavoidable due to reflections from the frame (101) or other objects. However, the frame (101) and/or drape (201) may be designed to be EMR absorptive in the relevant wavelengths being used or may be coated with an absorptive coating to inhibit reflection of the EMR.

In an alternative embodiment, the EMR radiators (301) may be aimed downward toward the patient (403). This will typically only be done with light which is unable to penetrate or cause irreparable damage to human skin or internal structures. This may be in the UVC range and will typically be at some of the shortest wavelengths (from 200-210 mm). It may also fall into the UV vacuum range (smaller than 200 nm). In this case, the light wall (303) may be used to bathe the incision (403) and underlying structures in EMR.

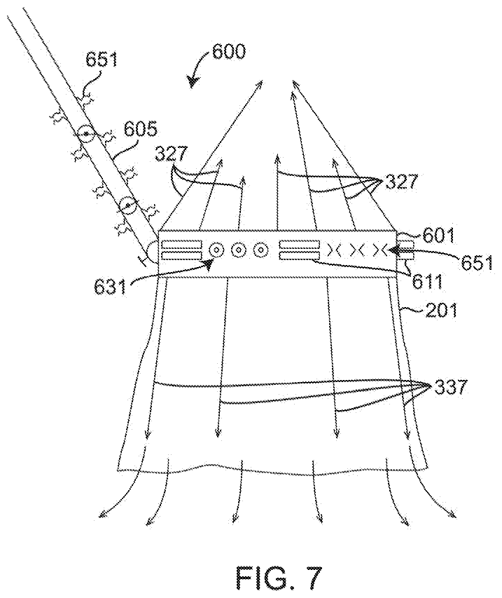

Regardless of the specifics of the EMR generation and if the light wall (303) forms a plane above the patient (401) or illuminates the incision (403) directly, the second sanitization object that will be used is at least one, and typically a plurality of, lateral air projectors (307) or “air knives”. These projectors (307) (of which there are four shown in FIG. 1 one on each side of the frame (101)) will be of insufficient strength to cut human flesh, but will serve to generate generally laminar flows (317) and (327) over at least a portion, and preferably all the internal periphery of the frame (101). These laminar flows (317) and (327) will typically be aimed upward relative to the frame (101) as shown in FIGS. 2 and 3. In FIG. 3, as well as FIG. 7, the flow (327) is also arranged to point inward relative to the inner periphery of the frame, forming a rough cone, while in FIG. 2 the flow (317) is more outward, forming a rough inverted conical frustum. The flows (317) and (327) will often not be arranged at a particularly steep angle, but will generally be arranged so as not to produce a single lateral (or planar) flow generally parallel to the major dimension of the frame (101), but instead produce multiple intersecting flows.

As part of the airflow, air will also be typically pushed downward from the frame (101) and into or across the surface of the drape (201) from inside the opening (103). This will serve to provide a slight positive pressure behind the drape (201) and provide flow (337) under a bottom edge (211) of the drape (201) as shown in FIGS. 2, 3, and 7. where it contacts the patient (401). Thus, there will be a structure of flow around and above the system (100) with the enclosed volume (501) under the frame (101) and within the drape (201) maintained at a slight vacuum or more specifically with a negative pressure serving to pull air into the volume (501) only via the opening (103). Thus, air pulled into the volume (501) will typically have to pass through the light wall (303).

It should be recognized that the flow from these projectors (307) does not need to be particularly powerful in many embodiments. The force will instead typically be sufficient to keep dust and aerosolized particulates in surrounding air from entering the opening (103) in the frame (101) from above and to similarly inhibit air movement (such as from wind) from getting around or under the drape (201). The flows (317) and (327) will also preferably have sufficient force to inhibit typical flying insects, particularly from those commonly attracted to blood or other body fluid, from being able to enter the opening in the frame (101) from above. However, the flow will typically be weak enough that it does not present an impediment or discomfort for a human to operate at the incision with their hands with the flows impacting their hands or forearms during the operation.

Air for the projectors (307) will typically be provided through the arm (105) if present from a compressor (503) which will be located toward the distal end (511) of the ann separate from the proximal end (513) at the frame (101). The compressor (503) will typically provide pressurized air to the frame (101) to form the air flows (317) or (327) through the projectors (307). The air will typically be filtered and cleaned prior to being provided to the frame (101) such as through the use of traditional filters (including HEPA filters) or through separate cleaning or sterilization systems including EMR and related sterilization systems.

The arm (105) may also be used as a conduit for electricity for use by the frame to power the EMR radiators (301) and/or other onboard systems which may include power outlets for connection of powered surgical tools or additional lights. Specifically, the arm (105) at the distal end (511) may be connected to a standard alternating current (AC) power outlet via a plug (505). In an alternative embodiment, either the frame (101) directly, the air, or related structures may be provided with a direct current (DC) power source including, but not limited to, chemical batteries, solar panels, or charged capacitors.

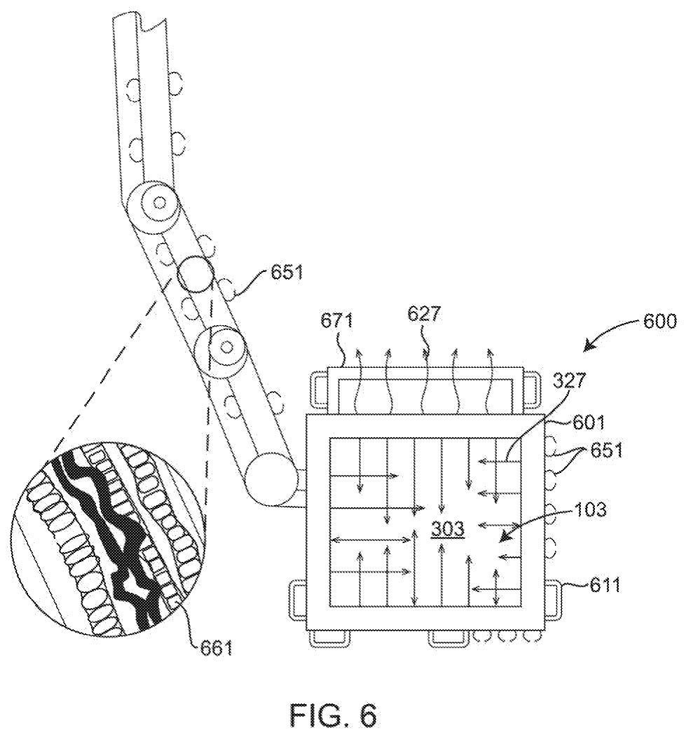

In the embodiment of FIGS. 6 and 7, the device (600) is designed to include further elements on the frame (601) and arm (605). In particular, both the frame (601) and arm (605) include spring clamps (651) or similar devices designed for cord management which can be useful if powered surgical tools are being used. Further, power and other outlets (621) can also be provided on the frame (601) to power these devices, supply them with air or water, or to otherwise supply them with necessary inputs. Similarly, outlets (621) may be designed to provide for vacuum suction for tools with the waste being exhausted to a location spaced from the frame (601) as is standard and understood in medical suction. All of these options may be provided in addition to or instead of handles (611) as previously contemplated.

In order to provide for air, water, power, and/or suction to be available at outlets (621), the arm (601) may include a number of pipes, tubes, or other conduits (661) for supplying these inputs (or outputs in the case of suction and waste disposal) to the frame (601). The embodiment of FIGS. 6 and 7 also further contemplates the inclusion of a tool tray (671) which may be attached to the side of the frame (601) or may be moveable or detachable relative to the frame (601). The tray may be of similar material to the frame and designed to have previously sterilized instruments and tools placed thereon. In the depicted embodiment, air flow (627) may be provided to flow across the instruments placed on the tray (671). The tray (671) may also be illuminated by the EMR from EMR radiators (301) or may include its own EMR radiators to provide for EMR disinfection of anything placed thereon.

In use, the system (100) will typically operate as follows and as illustrated best in FIGS. 4 and 5. The patient (401) would be provided upon a surface (107) and will either be under the effects of general anesthesia, restrained from movement, or instructed to simply keep still depending on the nature of the patient (401), the wound (403) to be acted on, and the circumstances surrounding the care. Local anesthetic may be applied to a wound (403) if appropriate and available.

For ease of discussion, the remainder of the operation will be discussed on the assumption hat the patient is under the effects of general anesthesia and that the wound (403) is an incision related to surgery. Use of the device in existing wound care will typically be generally similar.

The incision (403) area will typically be placed under the frame (101) and the drape (201) positioned to allow the system (100) to effectively surround the location of the incision (403) within the enclosed area (501). The frame (101) may then be secured in place as appropriate and depending on the embodiment of system (100) used. The system (100) will typically be activated once positioned and locked into position with both the projectors (307) forming the air flows (317) or (327) and the EMR radiators (301) forming the light wall (303) at around the same time and prior to the incision (403) being made. However, in alternative embodiments one or the other of the projectors (307) or EMR radiators (301) may be used alone.

Once the system (101) is active, the light wall (303) will typically be present across the opening (103) and the projectors (307) will be pushing air away from the opening (103) with flow (317) or (327). Standard disinfection of the patient's skin will typically occur at this time by reaching through the opening (103). Further, additional methods of disinfection may be performed on air which is currently under the frame (101) and above the patient (401) in enclosed area (501). This can include additional EMR exposure or other procedures. However, in an embodiment, the projectors (307) can serve to push air from this region (501) using the flow (337) and providing supplemental replacement air via the same process.

After the patient's (401) skin and the air inside the operational volume (501) has been disinfected as appropriate, the surgeon will typically perform all appropriate methods to disinfect their hands. This will often also involve them donning surgical gloves. The surgeon will then typically place their hands through the opening (103) from above and perform all action on the patient (401) while the device (100) is running. Their hands, thus, will pass through the air flows (327) or (317) and the light wall (303) immediately before and after they contact the patient (401) every time they move their hands through the opening (103). This will serve to disinfect and push away particles which may be on their hands and on any instruments they are carrying and using.

Upon completion of the procedure, the patient's incision (403) will be closed and the device (100) will be turned off and removed form over the patient (401). Between procedures the device (100) will be cleaned and disinfected in the same manner as other fixtures and/or tools utilized in surgery. The drape (201) may be removable to allow it to be separately washed and the frame (101) and arm (105) may be cleaned with disinfecting solutions. The EMR radiators (301) may also be used to disinfect the drape (201) by passing the drape (201) through the opening (103) when the lights (301) are on. This procedure may be used when other cleaning options are not available and the device (100) needs to be used on a new patient (401)

The embodiment of the device (800) of FIGS. 8-10 is designed to be even more portable and would typically be smaller and lighter than the other embodiments contemplated herein. In device (800), the frame (801) provides the light wall (303) in the same basic fashion as other devices (100) and (600). However, in device (800) portions of the frame (801) are designed to rotate relative to each other so as to make the sides generally parallel or collinear as shown in FIG. 10. The frame (801) elements may then also be rotated so that the lights (301) are aimed downward (into the page of FIG. 10). This allows the device to be used as EMR wand to that it can be used to disinfect a surface or to disinfect the patient themselves as appropriate.

While the above described a preferred operation and use of the device (100) as a surgical site barrier, the nature of the device (100) and its portability can allow for it to be put to alternative uses in certain situations. For example, in some circumstances, the device (100) could also be used to sterilize a wound directly. For example, under battlefield conditions where a major wound to, or amputation of, an extremity is common, the wound may require immediate closure to prevent death from bleeding and that the closure be performed in combat and within extraordinarily unsanitary conditions. Further, the action which caused the wound, such as an explosion, may have positioned unsanitary objects such as shrapnel or dirt in the wound area.

In this type of circumstance, the device (100) may be positioned in such as way so as to directly illuminate the wound with the EMR and/or push away material around the wound area. In the case of an amputation for instance, the remaining stump may be passed through the opening (103) (typically from underneath) while the device (100) is running. This will serve to directly bathe the wound in EMR as the wound passes through the light wall (303) and can also use the flows (317), (327), and/or (337) to push material away from the wound. A similar procedure may be used for a major wound so long as the location of the wound can fit through the opening (103). This type of use would generally not be preferred, but may be relevant in certain circumstances where damage done to the wound by EMR exposure form the light wall (303) and/or the pressure imparted by the flows (317), (327), and/or (337) may be considered secondary to the likelihood of severe injury or death from infection of the wound if it is not so treated.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

It will further be understood that any of the ranges, values, properties, or characteristics given for any single component of the present disclosure can be used interchangeably with any ranges, values, properties, or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

Finally, the qualifier “generally,” and similar qualifiers as used in the present case, would be understood by one of ordinary skill in the art to accommodate recognizable attempts to conform a device to the qualified term, which may nevertheless fall short of doing so. This is because terms such as “rectangular” are purely geometric constructs and no real-world component is a true “rectangular” in the geometric sense. Variations from geometric and mathematical descriptions are unavoidable due to, among other things, manufacturing tolerances resulting in shape variations, defects and imperfections, non-uniform thermal expansion, and natural wear. Moreover, there exists for every object a level of magnification at which geometric and mathematical descriptors fail due to the nature of matter. One of ordinary skill would thus understand the term “generally” and relationships contemplated herein regardless of the inclusion of such qualifiers to include a range of variations from the literal geometric or other meaning of the term in view of these and other considerations.

1. A surgical site barrier comprising:
a frame, the frame having an outer peripheral edge and an inner peripheral edge, the inner peripheral edge defining an opening through the frame;
a source of electromagnetic radiation (EMR) positioned on the frame; and
a drape positioned along the outer peripheral edge of the frame;
wherein, the source of EMR produces a generally planar illumination forming a light wall across the opening.

2. The barrier of claim 1, wherein the light wall is within the opening.

3. The barrier of claim 1, wherein the light wall is below the opening.

4. A surgical site barrier comprising:
a frame, the frame having an outer peripheral edge and an inner peripheral edge, the inner peripheral edge defining an opening through the frame;
a plurality of air projectors positioned on the frame; and
a drape positioned along the outer peripheral edge of the frame;
wherein, the air projectors produce intersecting generally laminar airflows which are non-parallel to the major dimension of the frame.

5. The barrier of claim 4, wherein the airflows intersect over the opening.

6. The barrier of claim 4, wherein the airflows intersect outside the inner periphery of the frame.

7. The barrier of claim 4 further comprising an airflow from the frame which passes under an edge of the drape.

8. A surgical site barrier system comprising:
a patient having an incision site;
a surgical site barrier device positioned over the incision site, the surgical site barrier comprising:
a frame, the frame having an outer peripheral edge and an inner peripheral edge, the inner peripheral edge defining an opening through the frame;
a plurality of air projectors positioned on the frame;
a source of electromagnetic radiation (EMR) positioned on the frame; and
a drape positioned along the outer peripheral edge of the frame;
wherein, the source of EMR produces a generally planar illumination forming a light wall across the opening; and
wherein, the air projectors produce intersecting generally laminar airflows which are non-parallel to the major dimension of the frame.

9. The system of claim 8, wherein the light wall is within the opening.

10. The system of claim 8, wherein the light wall is below the opening.

11. The system of claim 8, wherein the airflows intersect over the opening.

12. The system of claim 8, wherein the airflows intersect outside the inner periphery of the frame.

13. The system of claim 8 further comprising an airflow from the frame and under an edge of the drape.

14. The system of claim 8 further comprising an adjustable aim attached to the frame at a proximal end, the adjustable arm positioning the frame over the incision site.

15. The system of claim 14 wherein air and electricity for the air projectors and EMR radiators is provided via the arm.

16. The system of claim 15 further comprising an air compressor located at a distal end of the arm, opposite the proximal end, for suppling air to the air projectors.

17. The system of claim 16 wherein the supplied air is filtered prior to being supplied to the air projectors.

18. The system of claim 14 wherein the arm is connected to an alternating current (AC) power source.

19. The system of claim 8 further comprising a direct current (DC) power source.

20. The system of claim 8 wherein said EMR is UVC light.
